(19) United States
(12) Plant Patent Application Publication
(10) Pub. No.: US 2008/0148450 P1
(43) Pub. Date: Jun. 19, 2008

(54) CALIBRACHOA PLANT NAMED 'CALTRAELBU'

(75) Inventor: Johannes Simon Nicolaas Oud, Bovenkarspel (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/639,528

(22) Filed: Dec. 15, 2006

(51) Int. Cl. A01H 5/00

(52) U.S. Cl. PLT/413

(57) ABSTRACT

A new Calibrachoa plant particularly distinguished by its trailing, outwardly spreading and mounded plant habit, freely branching habit, freely flowering habit, single flowers that are burgundy in color, and good garden performance.

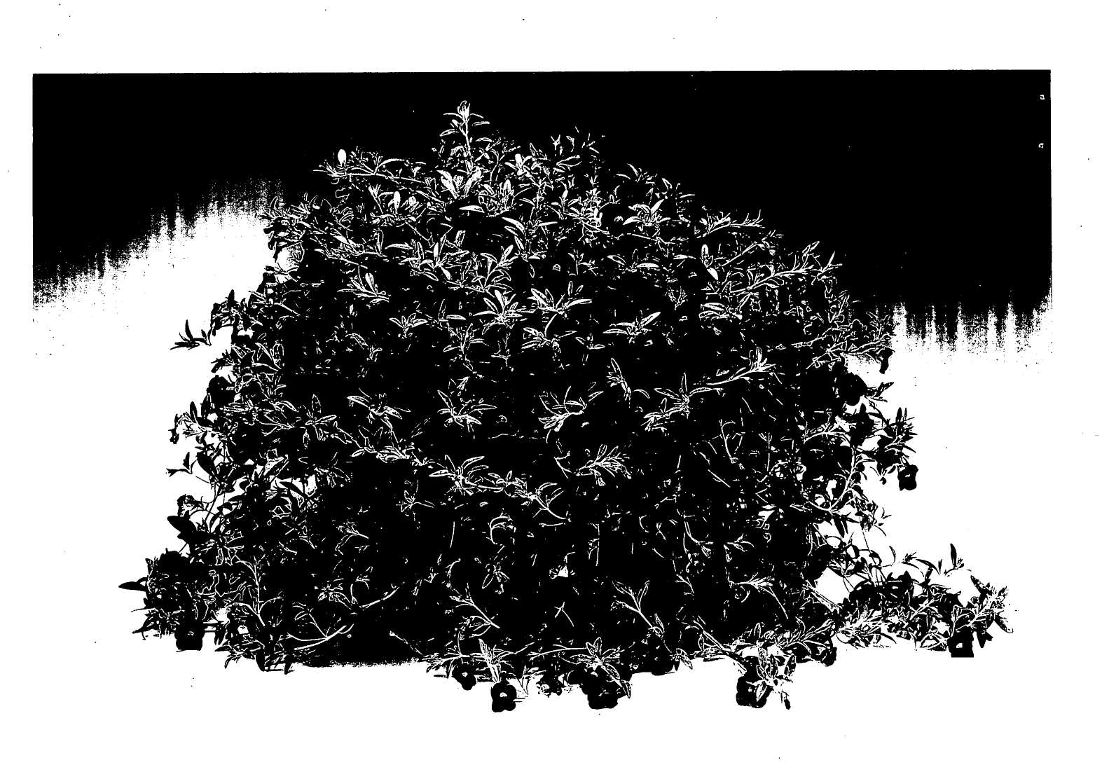

### LATIN NAME OF THE GENUS AND SPECIES OF THE PLANT CLAIMED

Calibrachoa hybrida

### VARIETAL DENOMINATION

‘Caltraelbu’

### BACKGROUND OF THE NEW PLANT

The present invention relates to a new and distinct cultivar of Calibrachoa plant botanically known as Calibrachoa hybrida and referred to by the cultivar name ‘Caltraelbu.’

The new Calibrachoa is the product of a planned breeding program conducted by the inventor in Enkhuizen, Netherlands. The new Calibrachoa cultivar is freely flowering with trailing and spreading plant habit and attractive leaf and flower coloration.

The new Calibrachoa originated from a cross-pollination made by the inventor in July 2003 of a proprietary selection of Calibrachoa hybrida identified as code number ‘Z4012,’ not patented, as the female, or seed parent, with the Calibrachoa hybrida variety ‘KLEC01055’ (U.S. Plant Pat. No. 14,171), as the male, or pollen parent. The new Calibrachoa was selected as a single plant from the resulting progeny of the cross-pollination in a controlled environment in Enkhuizen, Netherlands in May 2004.

Asexual reproduction of the new cultivar by termninal vegetative cuttings since August 2004, in Enkhuizen, Netherlands has shown that the unique features of this new Calibrachoa are stable and reproduced true to type in successive generations.

### SUMMARY OF THE INVENTION

Plants of the cultivar ‘Caltraelbu’ have not been observed under all possible environmental conditions. The phenotype may vary somewhat with variations in environment such as temperature, light intensity and daylength without, however, any variance in genotype.

The following traits have been repeatedly observed and are determined to be the unique characteristics of ‘Caltraelbu.’ These characteristics in combination distinguish ‘Caltraelbu’ as a new and distinct cultivar:

1. Trailing, outwardly spreading and mounded plant habit

2. Freely branching habit

3. Freely flowering habit

4. Single flowers with burgundy flowers

5. Good garden performance

In side-by-side comparisons conducted in Enkhuizen, Netherlands, plants of the new Calibrachoa differed from plants of the female parent selection in the following characteristic:

1. Plants of the new Calibrachoa have slightly more vigour than the plants of the female parent selection.

In side-by-side comparisons conducted in Enkhuizen, Netherlands, plants of the new Calibrachoa differed from plants of the male parent selection in the following characteristic:

1. Plants of the new Calibrachoa have a more trailing habit whereas the plants of the male parent selection are more upright.

TABLE 1
DIFFERENCES BETWEEN THE NEW CULTIVAR
‘CALTRAELBU’ AND A SIMILAR CULTIVAR

| | | Million Bells Cherry Red |
|---|---|---|
| | ‘Caltraelbu’ | ‘Sunbel-richpi’ (Not patented) |
| Plant habit | More trailing | Much more upright |
| Flower shape | A lobed flower | A more round flower |
| Throat width | wider | More narrow |

### DESCRIPTION OF THE DRAWING

The accompanying colored photograph illustrates the overall appearance of the new cultivar, showing the colors as true as it is reasonably possible to obtain in colored reproductions of this type. Colors in the photograph may differ slightly from the color values cited in the detailed botanical description which accurately describe the colors of the new Calibrachoa.

### DESCRIPTION OF THE NEW CULTIVAR

The observations and measurements describe plants grown in Enkhuizen, The Netherlands, practice circumstances outdoor in window boxes during the Summer with day temperatures ranging from 14 to 30° C., night temperatures ranging from 10 to 20° C. Plants used for the photographs and description were about 10 weeks from planting rooted cuttings.

In the following description, color references are made to The Royal Horticultural Society Colour Chart, 2001 Edition, except where general terms of ordinary dictionary significance are used.
The plant:
   Classification—botanical.—Calibrachoa hybrida.
Parentage:
   Female parent.—Proprietary selection of Calibrachoa hybrida identified as code number ‘Z4012,’ not patented.
   Male parent.—Calibrachoa hybrida variety ‘KLEC01055,’ U.S. Plant Pat. No. 14,171.
Propagation:
   Type cutting.—Terminal vegetative cuttings.
   Time to initiate roots.— Summer and Winter: About 12 days at 20° C.
   Time to develop roots, summer.—About 21 days at 200 C.
   Time to develop roots, winter.—About 28 days at 16° C.
   Root description.—Fine, fibrous, white in color.
   Rooting habit.—Freely branching.
Plant description:
   Form.—Annual flowering plant; indeterminate; initially upright, then trailing and outwardly spreading; uniformly mounded plant form. Freely branching habit; about six to eight basal branches each with about six to eight lateral branches. Pinching enhances development of lateral branches.
   Usage.—Appropriate for hanging baskets, window boxes, patio containers, and landscape applications.
   Plant height (from soil level to top of plant plane).—About 15 cm.
   Plant diameter (from soil level to lateral branches apices).—About 50 cm.
   Vigor.—Vigorous; rapid growth rate.
The stem:
   Main branches, length.—About 45 cm.
   Main branches, diameter.—About 2.2 mm.
   Lateral branches, length.—About 10 cm.
   Lateral branches, diameter.—About 1 mm.
Internodes:
   Length.—2 cm.
   Texture.—Pubescent.
   Strength.—Strong. Wiry.
   Color.—144A with some anthocyanin N187A.
Foliage description:
   Arrangement.—Alternate before flowering; opposite after flowers develop; simple; sessile.
   Length.—About 3.1 cm.
   Width.—About 9 mm.
   Shape.—Oblanceolate to elliptic.
   Apex.—Obtuse.
   Base.—Attenuate.
   Margin.—Entire.
   Aspect.—Mostly flat.
   Texture, upper and lower surfaces.—Slightly pubescent.
   Venation pattern.—Pinnate.
   Color.— Developing foliage, upper surface: 146A Developing foliage, lower surface: 146B Fully expanded foliage, upper surface: 146A, midvein 146B, lateral veins similar to lamina Fully expanded foliage, lower surface: 146B, midvein 146C, lateral veins similar to lamina.
The inflorescence:
   Flower type and habit.—Single salver form flowers; flowers face mostly upright or outward; axillary. Freely flowering habit.
   Natural flowering season.—Long day responsive; long flowering period, spring until frost in the autumn; flowering continuous during this period. Plants start flowering about nine weeks after planting rooted cuttings.
   Flower longevity on the plant.—About seven to ten days.
Flower size:
   Diameter: About 2.7 cm.
   Depth (height).—About 2.3 cm.
   Tube length.—About 1.5 cm.
   Throat diameter, distal end.—About 7 mm.
   Tube diameter, proximal end.—About 2 mm.
Flower buds:
   Length.—About 1.8 cm.
   Diameter.—About 4 mm.
   Shape.—Oblong.
   Color.— Towards the base: 150C Mid-section: 153D Towards the apex: 79A.
Petals:
   Arrangement/appearance.—Single whorl of five petals fused into flared trumpet.
   Length from throat.—About 1 cm.
   Width.—About 1.1 cm.
   Shape.—Roughly spatulate with a rounded apex.
   Margin.—Entire.
   Texture, upper and lower surfaces.—Smooth, glabrous, satiny.
   Color.— When opening, upper surface: N66A When opening, lower surface: 72C Fully opened, upper surface: N66A Circumorbital ring: N77A Venation, upper surface: 61B Fully opened, lower surface: 72C Venation, lower surface: 61B Flower throat (inside): 9B Venation, throat 61B Flower tube (outside): 4C Venation, tube: 61B.
Sepals:
   Arrangement/appearance.—Single whorl of five sepals fused at base, star-shaped.
   Length.—About 1.2 cm.
   Width.—About 3 mm.
   Shape.—Lanceolate.
   Apex.—Acute.
   Margin.—Entire.
   Texture, upper and lower surfaces.—Pubescent.
   Color, upper and lower surfaces.—144A.
Peduncles:
   Length.—About 9 mm.
   Width.—About 1 mm.
   Strength.—Flexible. wiry.
   Texture.—Pubescent.
   Color.—144A with some anthocyanin N187A.
Reproductive organs:
   Stamens, quantity per flower.—Five.
   Anther shape.—Four-parted, ovate.
   Anther length.—About 1.2 mm.
   Anther diameter.—About 1.1 mm.
   Filament length.—About 6 mm.
   Filament color.—1 50C.
   Anther color.—5C.
   Pollen amount.—Moderate.
   Pollen color.—5C.
   Pistils, quantity per flower.—One.
   Pistil length.—About 9 mm.
   Stigma shape.—Ovate.
   Stigma color.—144B.
   Style length.—About 7 mm.
   Style color.—145B with anthocyanin N I 87A.
   Ovary color.—145B; Apex N187A.
Seed development: Seed development has not been observed to date.
Disease and pest resistance: Plants of the new Calibrachoa have not been noted to be resistant to pathogens or pests common to Calibrachoa.
Garden performance: Plants of the new Calibrachoa have been observed to have good garden performance. Plants of the new Calibrachoa have been noted to tolerate rain, wind, and temperatures from 0 to 30° C.

What is claimed is:

1. A new and distinct variety of Calibrachoa plant, substantially as illustrated and described herein.
